(19) United States
(12) Patent Application Publication
(10) Pub. No.: US 2017/0101471 A1
(43) Pub. Date: Apr. 13, 2017

(54) BLOCKADE OF TIM-1 FOR TREATING GRAFT-VERSUS-HOST DISEASE

(71) Applicant: The Board of Trustees of the Leland Stanford Junior University, Stanford, CA (US)

(72) Inventors: Panagiota Iliopoulou, San Carlos, CA (US); Rosemarie H. Dekruyff, Portola Valley, CA (US); Everett Hurteau Meyer, Reston, VA (US)

(21) Appl. No.: 15/287,365

(22) Filed: Oct. 6, 2016

Related U.S. Application Data
(60) Provisional application No. 62/239,098, filed on Oct. 8, 2015.

(51) Int. Cl. C07K 16/28

(52) U.S. Cl. CPC C07K 16/2803; C07K 2317/76; A61K 2039/505

(57) ABSTRACT

TIM1 antagonists reduce GVHD by symptom score and show statistically significant improved survival.

CROSS REFERENCE

This application claims benefit of U.S. Provisional Patent Application No. 62/239,098, filed Oct. 8, 2015, which application is incorporated herein by reference in its entirety.

### FEDERALLY SPONSORED RESEARCH AND DEVELOPMENT

This invention was made with Government support under contract A1054456 awarded by the National Institutes of Health. The Government has certain rights in the invention.

### BACKGROUND OF THE INVENTION

Graft versus Host Disease (GVHD) is a major debilitating and potentially lethal, side effect of bone marrow transplantation. It occurs when lymphocytes from the donor present in the bone marrow inoculums attack and destroy healthy tissues of the recipient.

Acute GVHD occurs in recipients of allogeneic HSC transplants (in 40% of HLA-matched sibling graft recipients and 80% of unrelated donor graft recipients). It causes fever, rash, hepatitis with hyperbilirubinemia, vomiting, diarrhea, abdominal pain (which may progress to ileus), and weight loss. Risk factors include HLA and sex mismatching; unrelated donor; older age of recipient, donor, or both; donor presensitization; and inadequate GVHD prophylaxis. Diagnosis is obvious based on history, physical examination, and liver function test results; conventional treatment is methylprednisolone 2 mg/kg IV once/day, increased to 10 mg/kg if there is no response within 5 days.

Chronic GVHD may occur by itself, develop from acute GVHD, or occur after resolution of acute GVHD. It typically occurs 4 to 7 mo after HSC transplantation (range 2 mo to 2 yr). Chronic GVHD occurs in recipients of allogeneic HSC transplants (in about 35 to 50% of HLA-matched sibling graft recipients and 60 to 70% of unrelated donor graft recipients). It affects primarily the skin (eg, lichenoid rash, scleroderma) and mucous membranes (eg, keratoconjunctivitis sicca, periodontitis, orogenital lichenoid reactions), but it also affects the GI tract and liver. Immunodeficiency is a primary feature; bronchiolitis obliterans similar to that after lung transplantation can also develop. Ultimately, GVHD causes death in 20 to 40% of patients who have it.

Approximately half of patients who survive beyond 100 days after allogeneic HCT develop cGvHD. The common sites of organ involvement include the mouth, eyes, skin, gastrointestinal tract, liver and lungs. Manifestations of cGvHD adversely impact patient quality of life and mortality. The three-year survival rates for patients with newly diagnosed “favorable” and “poor” risk cGvHD are approximately 80% and 40%, respectively. Further, only 50% of patients with cGvHD are able to discontinue immunosuppressive treatment within 5 years after its onset. Chronic immunosuppressive therapy increases post-HCT morbidity and mortality with a direct correlation between dose of post-HCT corticosteroid and non-relapse mortality (NRM;

The current treatments for GVHD are intravenously administered glucocorticoids, such as prednisone. The use of these glucocorticoids is designed to suppress the T-cell-mediated immune onslaught on the host tissues; however, in high doses, this immune-suppression raises the risk of infections and cancer relapse.

Therefore, there is still a great need for providing efficient therapeutic strategies targeting GVHD, with lower side effects.

### SUMMARY OF THE INVENTION

The present invention relates to a TIM-1 antagonist, for use for preventing or treating GVHD. In some embodiments the antagonist is an antibody. In some embodiments the GVHD is acute GVHD. In some embodiments the GVHD is chronic GVHD. In some embodiments the TIM-1 antagonist is an antibody. In some embodiments the antibody interferes with signaling by TIM-1. In some embodiments the antibody is human or humanized. Antibodies may be selected for low activation through the Fc receptor; or may be selected to be active in ADCC or ADCP. In some embodiments the antibody is a bivalent antibody. In some such embodiments a bivalent antibody is targeted to an antigen on a population of immune cells, including without limitation B cell markers, T cells markers, and NK cell markers.

In some embodiments the transplant recipient is subjected to myeloablative conditioning prior to transplantation and treatment with an antagonist of TIM-1. -Anti-TIM1 blockade could be used in the context of non-myeloblative HCT to promote sustained mixed chimerism and immune tolerance

The therapeutic methods of the invention promote immune tolerance and better immunity in the hematopoietic transplant setting. Post-transplant immune dyregulation of the thymus by donor immune cells can have a lasting effect on recipient immunity. The inflammatory effects of the HCT process contribute to dysregulated thymus function, which may be prevented by treatment with the methods of the present invention.

In some embodiments a population of cells for engraftment, which cell population comprises allogeneic hematopoietic stem cells, is contacted ex vivo with an effective dose antagonist of TIM-1, which contacting is performed prior to transplantation. In some such embodiments the population of cells for engraftment comprises an effective dose of a donor lymphocyte infusion. In some such embodiments, the methods further comprise administering an effective dose of one or more of Breg cells; Treg cells; and iNKT cells.

In some embodiments, an effective dose of a TIM-1 antagonist is administered to a transplant recipient. In some embodiments the administering is performed prior to transplantation. In some embodiments the administering is performed following transplantation, e.g. substantially simultaneously, after about 1 day, after about 2 days, after about 3 days, after about 4 days, after about 5 days, after about 6 days, after about one week, after about 2 weeks, after about 3 week, after about 4 weeks, or more. In some embodiment, administering to a transplant recipient is performed in combination with ex vivo treatment of the graft.

In some embodiments, treatment with a TIM-1 antagonist is combined with post-transplant cyclophosphamide prophylactic treatment to enhance protection against GVHD. In some embodiments the dose of cyclophosphamide is sufficient to induce apoptosis, e.g. including without limitations apoptosis of hematopoietic cells, e.g. lymphocytes, monocytes, etc. Alternatively or in combination, antagonism of TIM-1 is combined with corticosteroid treatment. In some embodiments, the combination of therapies provides for an effect that is synergistic with respect to administering either treatment as a monotherapy. In some embodiments, the TIM-1 antagonist is combined with, or administered concurrently with, apoptotic cells. In some embodiments the apopotic cells are leukocytes, which can be, without limitation, donor leukocytes in early stages of apoptosis. In a related embodiment, the TIM-1 antagonist is combined with, or administered concurrently with factors produced by apoptotic cells. In some embodiments, the combination of therapies provides for an effect that is synergistic with respect to administering either treatment as a monotherapy. In a related embodiment, an antagonist of TIM-1 is combined or administered concomitantly with the introduction of apoptotic cells or factors produced in apoptosis to promote immune tolerance in the setting of dysregulated immunity as in organ or islet transplantation, autoimmunity and atherosclerosis.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. Survival of mice following hematopoietic cell transplantation with total body irradiation conditioning with MHC mismatched donors. Mice treated with anti-TIM1 blocking antibody (3D10) showed reduced GVHD by symptom score (top panel) and show statistically significant improved survival (bottom panel). Mice treated with agonistic anti-TIM1 antibody (3B3) showed a worsening of GVHD symptom score (top panel) and a statistically non-significant trend towards improved survival.

### DETAILED DESCRIPTION OF THE EMBODIMENTS

### Definitions

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

General methods in molecular and cellular biochemistry can be found in such standard textbooks as Molecular Cloning: A Laboratory Manual, 3rd Ed. (Sambrook et al., CSH Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

By “comprising” it is meant that the recited elements are required in the composition/method/kit, but other elements may be included to form the composition/method/kit etc. within the scope of the claim.

By “consisting essentially of”, it is meant a limitation of the scope of composition or method described to the specified materials or steps that do not materially affect the basic and novel characteristic(s) of the subject invention.

By “consisting of”, it is meant the exclusion from the composition, method, or kit of any element, step, or ingredient not specified in the claim.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

As used herein, the term “prevention” refers to alleviating the disease or condition from occurring in a subject which has not yet been diagnosed as having it. As used herein, the term “subject” denotes a mammal, such as a rodent, a feline, a canine, and a primate. Preferably a subject according to the invention is a human.

The terms “individual,” “subject,” “host,” and “patient,” are used interchangeably herein and refer to any mammalian subject for whom diagnosis, treatment, or therapy is desired, particularly humans.

The hepatitis A virus cellular receptor 1 (HAVCR1/TIM-1), is a member of the TIM (T cell transmembrane, immunoglobulin, and mucin) gene family, which plays critical roles in regulating immune cell activity especially regarding the host response to viral infection. TIM-1 is also involved in allergic response, asthma, and transplant tolerance.

The TIM gene family was first cloned from the mouse model of asthma in 2001. Subsequently it was demonstrated that members of the TIM gene family including TIM-1 participate in host immune response. The mouse TIM gene family contains eight members (TIM-1-8) while only three TIM genes (TIM-1, TIM-3, and TIM-4) have been identified in humans.

TIM genes belong to type I cell-surface glycoproteins, which include an N-terminal immunoglobulin (Ig)-like domain, a mucin domain with distinct length, a single transmembrane domain, and a C-terminal short cytoplasmic tail. TIM-1 is preferentially expressed on Th2 cells and has been identified as a stimulatory molecule for T-cell activation.

Sequences of human and mouse TIM polypeptides are described in PCT publication No. WO 03/002722, in U.S. Pat. Nos. 6,066,498, 6,204,371, 6,288,218, 6,084,083, 6,414,117, and 6,562,343, and in U.S. Patent Publication Nos. 2003/0069196 and 2003/0124114, the teachings of which are hereby incorporated by reference in their entirety.

The human TIM-1 polypeptide is also disclosed as Genbank Deposit No. NP 036338 and the nucleic acid sequence of the cDNA is disclosed as NM_012206. The amino acid and nucleic acid (cDNA) sequences of mouse TIM-1 are disclosed as Genbank Deposit Nos. NP 599009 and NM_134248, respectively.

The amino acid and nucleic acid sequences of naturally occurring human allelic variants of TIM-1 are disclosed in U.S. Patent Publication No. 2003/0124114. Additional domains of human and mouse tim-1, such as the signal sequences, transmembrane domains and intracellular domains are described in Mcintire et. al., Nat. Immunol. (2001); 2(12):1109-16, incorporated herein by reference.

An antagonist of TIM-1 is an agent, e.g. an antibody, that blocks TIM-1 activity in a cell. An antagonist may particularly include antibodies that selectively bind to TIM-1 and inhibit, or block activity.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure.

The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation.

Any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, antibody sequence elements are humanized, primatized, chimeric, etc, as is known in the art.

Moreover, the term “antibody” as used herein, can refer in appropriate embodiments (unless otherwise stated or clear from context) to any of the art-known or developed constructs or formats for utilizing antibody structural and functional features in alternative presentation. For example, embodiments, an antibody utilized in accordance with the present invention is in a format selected from, but not limited to, intact IgG, IgE and IgM, bi- or multi-specific antibodies (e.g., Zybodies®, etc), single chain Fvs, Fabs, Small Modular ImmunoPharmaceuticals (“SMIPs™”), single chain or Tandem diabodies (TandAb®), VHHs, Anticalins®, Nanobodies®, minibodies, BiTE®s, ankyrin repeat proteins or DARPINs®, Avimers®, a DART, a TCR-like antibody, Adnectins®, Affilins®, Trans-bodies®, Affibodies®, a TrimerX®, MicroProteins, Fynomers®, Centyrins®, and a KALBITOR®. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]

In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Graft-versus-host disease (GVHD) is a common complication following an allogeneic tissue transplant. It is commonly associated with stem cell or bone marrow transplant. GVHD can also occur after a blood transfusion if irradiated blood products are not used.

As used herein, graft-versus-host-disease is divided into acute and chronic forms: the acute form of the disease (aGVHD) is normally observed within the first 100 days post-transplant, and is a major challenge to transplants owing to associated morbidity and mortality; the chronic form of graft-versus-host-disease (cGVHD) normally occurs after 100 days. The appearance of moderate to severe cases of cGVHD adversely influences long-term survival. This distinction is not arbitrary: acute and chronic graft-versus-host-disease appear to involve different immune cell subsets, different cytokine profiles and somewhat different host targets.

A “therapeutically effective amount” is intended for an amount of active agent which is necessary to impart therapeutic benefit to a subject. For example, a “therapeutically effective amount” is an amount which induces, ameliorates or otherwise causes an improvement in the pathological symptoms, disease progression or physiological conditions associated with a disease or which improves resistance to a disorder.

### Methods

In accordance with the present invention, a therapeutic composition of an antagonist of TIM-1 is administered as a therapeutic drug to a subject who has undergone a transplant comprising hematopoietic cells (bone marrow, peripheral stem cells, hematopoietic stem cells, etc.) and/or as a prophylactic drug to a transplant, for example bone marrow (BM) or peripheral stem cell donor cells, to treat or prevent the development of GVHD. One of skill in the art can determine the patients who would potentially benefit from a therapeutic agent that would reduce or prevent the development of GVHD. One of skill in the art can determine the therapeutically effective amount of the composition to be administered to a subject based upon several considerations, such as local effects, pharmacodynamics, absorption, metabolism, method of delivery, age, weight, disease severity and response to the therapy.

A further embodiment is treating, preventing or reducing the risk of developing graft-versus-host-disease by administering TIM-1 antagonist to the donor tissue or cells prior to transplantation into the recipient. It is envisioned that administering the composition to the donor tissue or cells will attenuate the immune cells in the donor and prevent the development of the immune response that is mounted against the recipient's tissue, thus preventing or attenuating GVHD.

The TIM-1 antagonist composition can also be administered parenterally, which includes, but is not limited to intradermal, subcutaneous, intramuscular, intraperitoneal, intravenous, intraarterial, intramyocardial, transendocardial, transepicardial, intrathecal, and infusion techniques. In addition, the TIM-1 antagonist can be contacted with the donor tissue or cells ex vivo, prior to transplantation.

In an embodiment of the present invention, the composition is administered in an effective amount to decrease, reduce, inhibit or abrogate GVHD and toxicity related to standard therapy. The amount of antibody in the composition may vary from about 1 ng to about 1 g, more preferably, 0.1 mg to about 100 mg.

Treatment regimens may vary as well, and often depend on the health and age of the patient. Certain types of disease will require more aggressive treatment, while at the same time, certain patients cannot tolerate more taxing regimens. The clinician will be best suited to make such decisions based on the known efficacy and toxicity (if any) of the therapeutic formulations.

In specific embodiments, the composition is given in a single dose or multiple doses. The single dose may be administered daily, or multiple times a day, or multiple times a week, or monthly or multiple times a month. A series of doses may be administered daily, or multiple times a day, weekly, or multiple times a week, or monthly, or multiple times a month.

The improvement is any observable or measurable improvement. Thus, one of skill in the art realizes that a treatment may improve the patient or subject's condition, but may not be a complete cure of the disease. In certain aspects, the composition is administered in an effective amount to decrease, reduce, inhibit or abrogate levels of an immune response against the recipient.

An improvement in GHVD is also any observable or measurable improvement. Thus, one of skill in the art realizes that a treatment may improve the patient or subject's condition, but may not be a complete cure of the disease. In certain aspects, the composition is administered in an effective amount to decrease, reduce, inhibit or abrogate levels of immune response from the donor's cells, tissue and/or organ against the host's tissues. GVHD can be acute or chronic or mild or severe. Improvements in acute symptoms include any of the following, for example, decrease skin rash, decrease diarrhea, increase in liver function, decrease in susceptibility to infection. Improvements in chronic symptoms include, but are not limited to decrease skin rash, decrease dermatitis, decrease hair loss, decrease liver damage, decrease dry eyes and mouth, decrease susceptibility to infections and decrease lung and/or gastrointestinal disorders.

In order to increase the effectiveness of oral administration of the composition of the present invention, it is desirable to combine these compositions with standard therapy. For example, known immunosuppressant agents can be used in combination with the composition of the present invention. Exemplary agents known to prevent organ rejection are T cell modifiers such as cyclosporine (Neoral™, Sandimmune™), prednisone (Novo Prednisone™, Apo Prednisone™), azathioprine (Imuran™), tacrolimus or FK506 (Prograf™), mycophenolate mofetil (CellCept™), OKT3 (Muromorab CO3™, Orthoclone™), ATGAM™ & Thymoglobulin™ or serine-threonine phosphatase calcineurin (CN) inhibitors. In specific embodiments, the standard or approved treatment of GVHD, which is high doses corticosteroids, primarily high-dose methylprednisolone, is used in combination with the composition of the present invention.

In addition to immunosuppressant other anti-rejection and/or anti-GVHD therapies can be used in combination with the methods of the present invention. For example, therapies for preconditioning and prophylaxis of GVHD include, but are not limited to total body irradiation, cytosine arabinoside, L-phenylalanine mustard, cyclophosphamide, etoposide, triethylene thiophosphoramide, antithymocyte globulin, bisulfan, tacrolimus, methylprednisolone, cyclosporin, or methotrexate. Experimental therapies for treatment of GVHD include, but are not limited to cytokine inhibitors/antagonists (e.g., anti-TNFα antibody); IL-1 receptor antagonist; recombinant IL-1 receptor; inhibitors of T-cell activation (e.g., tacrolimus); antimetabolites (e.g., mycophenolate mofetil); anti-CD3 antibody (muromonab, OKT3); anti-CD25 antibody; anti-IL2 receptor monoclonal antibody daclizumab; extracorporeal photopheresis using ex vivo 8-methoxypsoralen; anti-thymocyte globulins (Thymoglobulin™ or ATGAM™); ABX-CBL or CBL-1; or Visilizumab (Nuvion™).

The composition of the present invention may precede, be co-current with and/or follow the other agent(s) by intervals ranging from minutes to weeks. In embodiments where the composition of the present invention, and other agent(s) are applied separately to a cell, tissue or organism, one would generally ensure that a significant period of time did not expire between the time of each delivery, such that the composition and agent(s) would still be able to exert an advantageously combined effect on the cell, tissue or organism.

Various combination regimens of the composition and one or more agents are employed. One of skill in the art is aware that the composition of the present invention and agents can be administered in any order or combination.

A TIM-1 antagonist, e.g. an antibody that binds to and blocks the activity of TIM-1, may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, parenteral, intranasal, intravenous, intramuscular, subcutaneous or intraocular administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment. To prepare pharmaceutical compositions, an effective amount of the antibody may be dissolved or dispersed in a pharmaceutically acceptable carrier or aqueous medium. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

An antibody of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils.

The proper fluidity can be maintained, for example, by the use of a coating, such as lecithin, by the maintenance of the required particle size in the case of dispersion and by the use of surfactants.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride.

Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filtered sterilization.

Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose.

These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The antibodies of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered. In addition to the compounds formulated for parenteral administration, such as intravenous or intramuscular injection, other pharmaceutically acceptable forms include, e.g. tablets or other solids for oral administration; time release capsules; and any other form currently used.

In certain embodiments, the use of liposomes and/or nanoparticles is contemplated for the introduction of antibodies into host cells. The formation and use of liposomes and/or nanoparticles are known to those of skill in the art.

The invention will further be illustrated in view of the following figures and example.

### EXPERIMENTAL

TIM-1 is a surface receptor expressed on T cells, NKT cells, B cells, mast cells, and some epithelial cells of the kidney and lungs. The receptor binds to phosphatidylserine which is expressed on apoptotic cells, on exosomes, on some enveloped viruses and as a free floating product in the body.

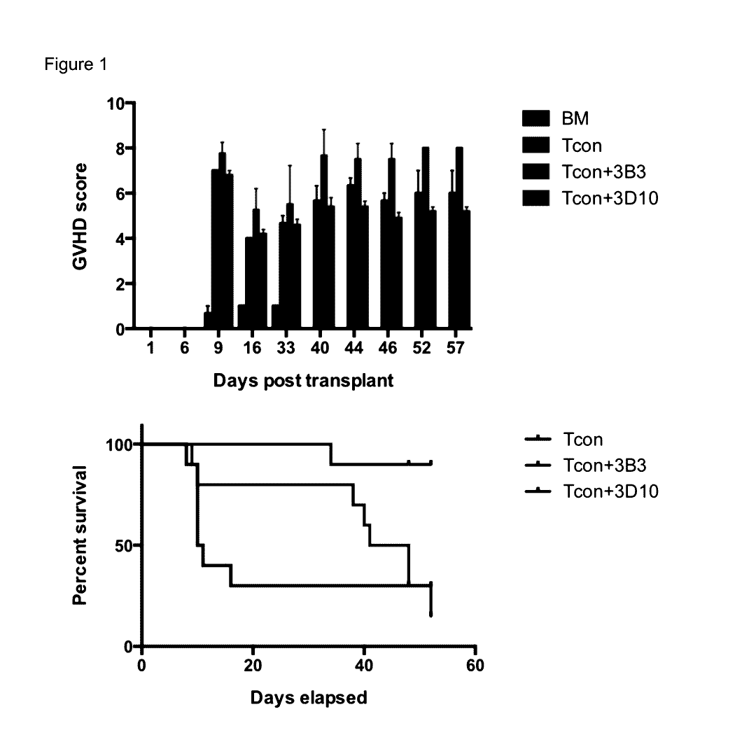

Treatment of recipient mice just prior to and following bone marrow transplantation with TIM-1 blocking antibody prevents GVHD (FIG. 1).

Our studies show that TIM-1 knock-out recipients show a modest survival advantage, pointing to a mechanism of action of TIM-1 blockade on a donor immune cell population.

What is claimed is:

1. A method for treating a graft versus host disease in a subject in need thereof, comprising administering to said subject a therapeutically effective amount of an antagonist of TIM-1.

2. The method of claim 1, wherein the antagonist is an antibody.

3. The method of claim 2, wherein the graft versus host disease is selected from the group consisting of acute graft-versus-host-disease (aGVHD) and chronic graft-versus-host-disease (cGVHD).

4. The method of claim 1, wherein graft cells are contacted with the TIM-1 antagonist prior to transplantation.

5. The method of claim 1, wherein the TIM-1 antagonist is administered following transplantation of the graft.
